(19) United States
(12) Patent Application Publication
(10) Pub. No.: US 2012/0101377 A1
(43) Pub. Date: Apr. 26, 2012

(54) Positron emission tomography (PET) imager of excised tissue specimens for cancer margins definition

(76) Inventors: Hannah Hazard, Morgantown, WV (US); Stanislaw Majewski, Morgantown, WV (US); Raymond Raylman, Morgantown, WV (US); James Proffitt, Newport News, VA (US)

(21) Appl. No.: 13/317,655

(22) Filed: Oct. 25, 2011

Related U.S. Application Data
(60) Provisional application No. 61/455,793, filed on Oct. 26, 2010.

(51) Int. Cl. A61B 6/00

(52) U.S. Cl. 600/436

(57) ABSTRACT

A first embodiment can be a device comprising a means to define cancer margin definition of a tumor resection specimen via radiotracer uptake using a table top, mobile 3D PET imager in the operating room.

### CROSS-REFERENCE TO RELATED APPLICATIONS

This patent application claims the benefit of the priority of provisional patent application 61/455,793.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BRIEF DESCRIPTION OF THE SEVERAL VIEWS OF THE DRAWING

Not Applicable

### DETAILED DESCRIPTION OF THE INVENTION

In the late 1800s, Halstead proposed breast cancer was a local disease and the best means of treating it was radical resection. So began the evolution of the surgical treatment of malignancies of the breast. In the 1970s, the National Surgical Adjuvant Breast and Bowel Project (NSABP) began trial B-04 which compared patients' overall survival of Halstead's radical mastectomy to total mastectomy with or without axillary dissection, depending on the clinical nodal status. The results of this study, with 25 year follow up data, indicate there is no difference in overall survival between the two groups of women. Shortly thereafter, NSABP B-06 opened; a trial comparing mastectomy to local excision with negative margins with and without post-operative radiation therapy. Again, 20 year follow up data indicates there is no difference in overall survival. With these results, women are more likely to choose breast conservation therapy (lumpectomy with negative margins followed by radiation therapy) over mastectomy when given the choice. The difficulty for breast surgeons becomes the removal of adequate tissue to achieve negative margins while not taking so much breast parenchyma to result in significant cosmetic deformities. Re-operation to achieve negative margins can occur up to 50% of the time resulting in increased anxiety, increased exposure to anesthetic complications, and increased risk of poor cosmesis. Despite attempts at intra-operative frozen section, intra-operative ultrasound and specimen radiographs, a high proportion of women require re-operation to clear the margins before they proceed to radiation therapy. This re-excision is one of the great frustrations to breast surgeons; devising an intra-operative detection system that defines the lesion in situ as well as defines the tumor ex vivo with clear understanding of the margins in three dimensions has the potential to reduce the aforementioned risks associated with breast cancer surgery but also the potential to improve our oncologic control of the primary tumor.

We are proposing a table top/mobile compact 3D PET imaging system, located in the operating room (OR), to provide fast in-situ determination of tumor resection and adequate surgical margins. While the final margin confirmation will be obtained from the pathology, such a device can reduce the total time necessary for the procedure and the number of interventions required to achieve satisfactory tumor resection with negative margins. It can also lead to a decrease in the number of additional surgeries. In breast cancer literature, up to 50% of lumpectomy patients require additional surgery to obtain negative margins; something that had not occurred with the primary surgery.

Positron Emission Tomography (PET) with F-18 Fluoro-deoxyglucose (FDG) has been shown to be a valuable tool for imaging various cancers because they demonstrate increased glucose metabolism as compared to normal tissues. When FDG is delivered systemically by intravenous injection, it concentrates in tumors allowing them to be detected and imaged through noninvasive means using PET imagers. Biomarkers other than FDG can be also used in surgical applications. For example FLT-F-18 and Choline-F-18 have been used in breast cancer and prostate cancer imaging, respectively. In this protocol, the patient will be injected with a sufficient amount of a positron emitting radiopharmaceutical, or radiotracer, such as FDG before surgery. When the lesion in the breast is non-palpable, patient will, at least initially, have a localizing wire placed under mammography guidance, to provide additional information about the location of the lesion location. Incidentally, wire localization does not provide information about the margins of the tumor, only localized the lesion in the breast.

Surgical resections of primary breast cancers are oriented by the surgeon upon removal from the breast. Subsequently, pathologists further orient the specimen with dye so the margin status can be determined during histiologic evaluation. With the addition of a set of miniature video cameras, we propose we will have the ability to determine the adequacy of those margins prior to pathologic evaluation. For example each of the imaging modules can have a camera mounted in the center of the module. The cameras will have only minimal effect on absorption of 511 ke V annihilation gammas, but, if necessary, can be removed for the PET imaging procedure.

Another method of marking the position of the lesion within the excised specimen could be based on radioactive markers that are placed within the volume or at the edges of the excised specimen. The radioactive markers, such as Na22, could be in the form of encapsulated seeds, or placed in the tips of needles. The markers would be imaged by the specimen PET imager at the same time or after the PET image(s) of the specimen will be acquired. The knowledge of the relative physical/visual positioning of the markers in the specimen tissue, and in the collected PET images, will provide guidance to the surgeon if the 3D physical margins around the lesion are sufficient, or a corrective additional excision is necessary.

In cases of low uptake of radioactively labeled biomarker in the normal tissue, such as fatty tissue, in order to enhance the visibility and position of the boundaries/edges of the excised specimens relative to the high uptake cancerous lesions, the whole specimens could be immersed in a container with a radioactive solution, before being imaged. The imaging procedure will then produce 3D image of the specimen in the container and containing the contrast liquid. The boundary between the specimen volume and radioactive solution will then become visible, or enhanced, on the reconstructed images providing better definition of the specimen edges and subsequently of the healthy tissue margins around the lesion(s). Other methods of enhancing the visibility of the specimen boundary can involve “painting” or spraying the specimen edges with radioactively labeled compound before imaging in the specimen in the PET imager. Alternatively, the specimen can be immersed and then removed from a container with a radioactively labeled compound. Some quantity of the compound will remain on the surfaces of the specimen. The compounds used in one of these surface enhancing procedures will have most likely higher viscosity than water, with examples such as gelatin or other glue-like compounds, for better adherence to the specimen's surface. In addition, these compounds can be colored or dyed with several different colors to provide additional visible directional orientation of the excised specimen relative to the excision site. This orientation is necessary to provide proper directional guidance in the excision cavity in cases when additional corrective excisions are necessary in order to achieve, or to increase negative surgical margins.

Yet another approach can use small radioactive seeds or other small particle type markers that are attached/glued etc. to the specimen sides or surfaces. The spacing or number of the seed type markers can be selected depending on the specimen size and complexity of its shape. Examples of seed markers are aerogel, polymer gel, or porous silica beads that are soaked with radioactive solution before being attached to the specimen's surface.

In its simplest form the Positron Emission Tomography (PET) Tissue Sample Imager uses a pair of rotating small flat PET cameras to image radiotracer uptake in the resected tissue sample. The cancerous tissue will typically be identified as a focal area or focal areas of increased radiotracer accumulation within the excised specimen. Other designs of the PET imager include four flat modules arranged in two pairs and, preferentially, a ring of several (typically 6-16) modules surrounding the specimen and providing the prompt 3D view of the focal uptake(s) in the specimen. Evaluating the PET images in at least two different 90 degree orientations or, preferentially images with 3D reconstruction, allows the surgeon to quickly determine if adequate margins have been obtained or, if further intervention is required. This process can be completed in just minutes in the operating room. The PET detector modules can be arranged in a vertical plane, with rotation in the same plane, and with “side loading” of the tissue specimens. In the second approach the specimens can be “top loaded” and placed between the PET detector modules arranged and rotating in the horizontal plane. The imager can be rotated on a gantry around the specimen by a limited angle (up to 30 deg) first in one direction and then in the opposite direction, to provide better 3D angular sampling of the imaged specimens. In another, mechanically simpler option, the specimen trey/bed rotates with the specimen on by the same limited angles, instead of the rotating detector ring.

Elements of the disclosed cancer margins PET imager:

One planar PET pair, two planar PET pairs, or ring PET imager with typically 10 cm×10 cm active field of view and ˜1.0-3.0 mm intrinsic spatial resolution
Rotation gantry (optional for ring PET)
Flexible support tray/bed for tissue specimens
OR-compatible mobile cabinet with shelves, and the PET imager on top, for housing electronics/computers
Optional arm with PET imager mount for easy close access
Computer(s) with monitor keyboard and mouse
Imager signal processing electronics with low voltage and high voltage power supplies
PET imager data acquisition system (hardware)
Data acquisition and processing software
Image reconstruction software, optionally on the second computer
Medical quality USB/isolation transformer to assure uninterrupted power during surgery and to provide electrical safety buffer
Optionally, a set of miniature video cameras can be attached for example to the PET imaging modules, to provide optical information helping with relative localization of active lesions, relative to the physical boundaries of the specimens

Several imaging technologies can be implemented in a proposed device. The preferred general type of a solution will have a scintillator as a sensor/energy converter of the 511 keV annihilation gamma rays, while different photodetectors will serve as detectors of the scintillation light produced by the absorbed 511 keV gamma rays in the scintillator gamma sensor. The scintillator sensor part can be made of pixellated or plate—continuous crystal scintillator materials such as LSO, LYSO, GSO, BGO, LaBr3, NaI(Tl), CsI(Tl), CsI(Na), and other. The photodetector part can be a standard or multi-element photomultiplier, position sensitive, flat panel or microchannel plate based photomultiplier, avalanche photodiode arrays or large size avalanche photodiodes with resistive etc readout, and different variants of the novel so-called Silicon Photomultiplier (SiPM). The SiPM solution is compact and can operate in magnetic fields, but it is comparatively expensive.

To read the signals from the flat PMT or SiPM based systems, intrinsically based on many individual pixels, readout reduction schemes can be implemented. For example a H8500 flat panel PMT has 64 individual anode channels, and H9500 has 256 anode pixels-channels. A channel-reducing readout scheme converts the intrinsically high granularity of basic readout elements (pixels), typically in the 3-6 mm range, to a more manageable number of readout sectors, lines, or strips, read then by a fast multi-channel readout system, such as FPGA-based USB2 fast data acquisition readout system available from Adaptive I/O Technologies, Inc. Such data acquisition systems can operate with different user-defined or commercial data acquisition software. Finally, there are different sources of 3D tomographic reconstruction algorithms that can be implemented to produce sectional images through the tissue specimen.

Examples of Preferred PET Imager Technologies:

In one possible implementation, the imager is based on two opposed detector heads, each made with an array of 2×2 Hamamatsu H8500 or H9500 2″ Flat Panel Photomultipliers, each PMT coupled to an array of 2 mm×2 mm×15 mm L YSO scintillators and forming a planar PET imager with a ˜10 cm×10 cm active field of view; the module pair will be rotated to provide different angular views of the specimen
In the second implementation, two pairs of planar and opposed imaging modules, as described above, will provide two simultaneous 90 degree views of the specimen; the two module pairs can be rotated to provide better sampling of angular views to allow for a 3D tomographic image reconstruction of the specimen
In the preferred implementation based on the already well-proven but novel and compact technology, a ring of 6 modules each made from two H8500 or H9500 PMTs, can be assembled to provide static tomographic imaging of the specimen under study; small imager rotation maybe still added to assure an even better a 3D tomographic image reconstruction of the specimen
In a more compact, light-weight implementation, the above flat panel PMTs can be replaced with new Silicon Photomultipliers (SiPMs). Such devices are for example available from Hamamatsu, SensL and other companies, and were demonstrated to be able to replace PMTs in several pilot applications
Similar fast on-board readout and multi-channel data acquisition systems can be used in both types of devices

What is claimed is:

1. A device comprising a means to define cancer margin definition of a tumor resection specimen via radiotracer uptake or by placed radioactive markers using a table top, mobile 3D PET imager in the operating room.

2. The device of claim 1 wherein the imager is comprised of one or more pairs of small flat PET camera modules arranged to rotate around a specimen bed.

3. The device of claim 1 wherein the imager is comprised of one or more pairs of small flat PET camera modules arranged in a ring around a rotating specimen bed.

4. The device of claim 1 wherein the imager is comprised of small flat PET camera modules arranged in a vertical rotational plane with the specimen loaded horizontally from the side.

5. The device of claim 1 wherein the imager is comprised of small flat PET camera modules arranged in a horizontal rotational plane with the specimen loaded vertically from the top or bottom.

6. The device of claim 4 wherein the rotational plane of the modules can be angled side to side up to 30 degrees from the vertical plane containing the specimen.

7. The device of claim 5 wherein the rotational plane of the modules can be angled up and down up to 30 degrees from the horizontal plane containing the specimen.

8. The device of claim 2 wherein the modules can rotate in both directions.

9. The device of claim 3 wherein the rotating specimen bed may be angled up or down up to 30 degrees from the plane of the modules.
